(19) United States
(12) Patent Application Publication
(10) Pub. No.: US 2008/0251551 A1
(43) Pub. Date: Oct. 16, 2008

(54) Medication Dispenser and Carrier Therefor

(75) Inventors: Ben Huber, Clayfield (AU); David Peter Wharton, Bribie Island (AU)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: MEDI-STREAM PTY LTD, BOWEN HILLS (AU)

(21) Appl. No.: 12/064,617

(22) PCT Filed: Aug. 22, 2006
(86) PCT No.: PCT/AU2006/001212

§ 371 Date: Feb. 22, 2008

(30) Foreign Application Priority Data
Aug. 22, 2005 (AU) 2005904543

(51) Int. Cl. A61B 19/02; A61M 11/00

(52) U.S. Cl. 224/197; 128/200.23

(57) ABSTRACT

The specification describes a medication dispenser for receiving a pressurised medication container and discharging therapeutic doses therefrom. The medication dispenser comprises an elongate tubular body with a cavity formed in the body and adapted to receive a medication container. Lateral aperture is formed in the body and communicates with the cavity. A sleeve is rotatably mounted to a first end region of the medication dispenser. The sleeve has a sleeve aperture which moves into and out of alignment with the lateral aperture during rotation of the sleeve. A mouthpiece moves between a discharge position and a storage position with rotation of the sleeve. The tubular body has a longitudinal axis which deviates away from the position of the extended mouthpiece, the deviation occurring outside the first end region. The invention may extend to a carrier for use with the medication dispenser.

### FIELD

The present invention relates to a medication dispenser for receiving pressurised medication containers and storing them in safe and hygienic conditions while also allowing activation when required. The invention extends to a carrier for the medication dispenser, the carrier preferably adapted for fixing in safe position on a personal item such as a belt, a strap, a carry bag or other suitable location accessible by a user.

### BACKGROUND

The evolution of devices for self-medication has been of great advantage to sufferers of various diseases and pathologies.

One prime example is that of asthma. In earlier times, sufferers of asthma were typically sentenced to a lifetime of disability, often based on arranging their life around low levels of physical activity or restricted ranges of travel, so that they could be close to support mechanisms provided in their own environment. Severe bouts of asthma often required the attention of professional medical practitioners, such as doctors, and the use of drugs administered by those professionals. These drugs, which included aminophylline and adrenaline, had reasonable levels of efficacy but also brought with them some significant risks in use. Further, the use of these drugs was only ameliorating and usually did not provide any ongoing advantage to a patient in the way of control and stabilisation of the disease condition.

The development of corticosteroids was of great assistance in some diseases and, in particular, asthma. However, the side effects of prolonged oral or parenteral administration of corticosteroids in people are notorious and necessitate restrictions on the adoption of this approach in other than the most severe cases.

A dramatic improvement in the quality of life of sufferers of asthma arose with the development of pressurised medicated containers designed, in part, for the self-administration of salbutamol, a bronchodilator, which is very effective in counteracting the bronchospasm of an asthma attack. Salbutamol is usually provided in a pressurised container with a depression-activated valve at its top. The valve is configured to nest in a seat provided on an outer plastic collar, cap or mouthpiece. This collar allows the depression of the canister relative to a valve stem, thereby releasing a controlled dose of therapeutic agent into a discharge throat of the collar. A patient uses the device by exhaling to a required extent and then inhaling while depressing the canister to discharge a dose of the agent.

As well as providing the ability to treat an attack of respiratory embarrassment, salbutamol inhalers have provided the ability to self-administer a regime of treatment to thereby minimise clinical signs and allay or prevent further development of the respiratory attack.

Salbutamol is an excellent example of a suitable therapeutic agent for self-delivery but is by no means alone. Many other forms of therapy have been provided for asthma (for example, Becotide® Flexitide®, Asmol®). Some of these are high efficacy, low dose corticosteroids relatively free of adverse side effects. Additionally, many other diseases lend themselves to self-medication through the provision of a metered dose, either into the respiratory tract or for ingestion through the gastrointestinal tract or absorption through the mucous membranes of the oropharynx or the nose.

Provision of the medication may be in a form as described, being a compressed pressurised aerosol formulation often in a device known as metered-dose inhalers or MDIs. Alternatively, powder or other solid formulations or even liquids or gases may be provided and dispensed at a set dose from a pressurised container.

While these developments have been of tremendous benefit to sufferers of diseases that lend themselves to effective self-medication, there has been an ongoing problem of patients either forgetting their medication canisters or mistakenly believing they are stored somewhere, such as a handbag or sports bag, only to find the medication is not available for use when required. This can have serious consequences in the event of a sudden severe onset of disease signs and symptoms. The problem of effectively and safely storing medication canisters is exaggerated in sporting activities where clothes are often designed for the specific requirements of the sport being undertaken, but with no provision of secure pockets or pouches. Even when pockets are present, a medication dispenser carrier may be susceptible to damage through impact or introduction of contaminants. The problems may be pronounced in outdoor activities, such as snorkelling, scuba diving, orienteering and mountaineering where a sufferer of a condition may find themselves a considerable distance from a support base with little else other than gear required for the activity.

Use in harsh environments or inclement conditions, such as rain and snow, may lead to ingress of moisture and other contaminants, such as mud, dust, sand, vegetable matter or other materials that may damage a medication device or present a risk to the user.

U.S. Pat. No. 4,130,116 (“Cavazza”) describes a pocket device into which a spray can may be inserted. Mechanical means are provided wherein the device may be kept in a closed position when not in use but can be activated to expose the nozzle portion of the spray can to permit utilisation of the can. The disclosure is to two halves that slide with respect to each other along guides located along borders of the lateral walls. There is no indication that the device is sealed against moisture and, in its operation, a wide aperture is presented to the environment both at the first nozzle end and second actioning end. The application of the device is therefore somewhat limited particularly in inclement environments, such as are often encountered during sporting activities, particularly waterborne sports and endurance type activities.

CA 2,379,137 (“Pharmaceutical Discovery Corporation”) describes a dry powder inhaler having an intake section, a mixing section and a rotatable mouthpiece. The device is relatively complex, although it does include a storage section for holding an extra medicament capsule. The device is particularly directed towards controlling a rate of airflow using a tapered piston rod and spring and one or more bleed-through orifices. It may also include a feedback module to generate a tone indicating when a proper rate of airflow has been achieved. Again, there is no indication that this device is environmentally sealed. It appears to be relatively bulky and complex in operation which increases the tendency for malfunction in the presence of foreign matter. Further, a person in shock or respiratory distress may be unable to provide the necessary inspiratory effort to activate it.

U.S. Pat. No. 5,497,764 (“Ritson”) is directed to a portable battery powered handheld system for releasing a controlled dose of aerosol medication for inhalation by a patient. The device includes a durable body and a medication cassette inserted in the durable body. Although the disclosure is to an electronic and relatively complex apparatus, it does disclose formation of a cassette for insertion inside the device which is constructed so that it can be used as a conventional, manually actuated metered dose inhaler device apart from the durable body. This, however, requires removal of the device from the body for its function. When housed in the durable body, the operation of the device is relatively complex. The pharmaceutical formulations may be a liquid or powder formulation. The device is not environmentally sealed. It appears relatively complex and is predominantly electronic.

BE 905189 (“Glaxo Group Ltd”) is directed to a device for administering medicaments in solid finely divided form to patients. The device is relatively complex, having a housing and a tray with a support disk provided on the tray and adapted to receive a carrier which, in turn, carries the medication. A plunger is operable to penetrate the container after it is aligned with the plungers. Air enters through the device and is inhaled.

DE 4028387 (“Bechter”) discloses a cover device that dissembles to provide a breathing mask with free space over the nose and chin. It is therefore relatively large. It is designed in one embodiment as a breathing mask for use with an inhalation apparatus.

WO 01100263 (“Inhale Therapeutic System”) discloses an arrangement in which air is prevented from entering the lungs until a prescribed threshold of vacuum is obtained by a user. Air is then abruptly permitted to flow to the lungs. This may be contradicted in a subject in shock or respiratory distress.

WO 1995/028192 (“Dura Pharmaceuticals”) describes a dry powder medicine inhaler having a housing and a mouthpiece. An impeller is rotatably mounted on a pin to rotate within the aerosolizing chamber. Radial inlets pass through the housing and enter substantially tangentially into the chamber. A charging plunger is pressed downward in operation against the bias of a spring to press a full dose of powdered medicine into the chamber. The plunger is then held against the spring bias during operation and forms a top portion of the wall of the chamber. A dosage cartridge may be placed on pegs in an open area. A hold down lever is then pivoted to retain the cartridge and lock a mouthpiece in its operative position. A multi-dose medicine-containing cartridge may be used. The impeller is driven at high speed by a motor. The impeller acts as a centrifugal air pump drawing air through the inlet and is driven by a high speed electric motor which relies on one or a pair of batteries. Again this device relies on battery input. However, the device can be operated by inhalation from the user alone. The device does not appear to be environmentally sealed against moisture in particular. Further, in both embodiments, it is a relatively complex device and is restricted to use of powder.

WO 2000/018455 (“Glaxo”) provides an inhalation device suitable for dispensing respiratory medication. It has a cover to prevent build-up of dust on the mouthpiece but is not sealed against water. It also has a geared and relatively complex actuator for use in operation.

WO 2002/004043 (“Vapotronics”) discloses an inhaler to deliver medicament or other fluids in droplet form and during inhalation. The inhaler has an airflow conduit assembly extending through the housing, a mouthpiece which is fluidly interconnectable with the airflow conduit assembly and a droplet ejection cartridge housing. A droplet ejector cartridge is removably disposed within the cartridge housing. The airflow conduit assembly may include a plenum into which air is initially drawn. The mouthpiece is removable and stowable on the device. A droplet ejector cartridge is provided which includes a medicament reservoir, a plurality of droplet ejection orifices and at least one droplet ejection actuator. The droplet ejection cartridge includes a PCB interface on one of its sides for interfacing with a printed circuit board which controls the operation of the inhaler. Pressure sensors are used to activate the device and a nozzle region is provided which includes a plurality of droplet ejection orifices. Each droplet ejection orifice has its own resistor; so there is a one-to-one relationship for dispensing the medicament. The device is therefore a complex electronic arrangement and relies on electric power in use. It may have limited application in harsh environments.

UK Patent GB 2074454 (“Somova”) discloses an inhalation device with a retractable mouthpiece. This device has a tubular body adapted for detachable fitting to an aerosol container. A delivery mouthpiece is fitted which is movable between retracted and extracted positions. The extraction and retraction of the mouthpiece is brought about, at least in part, by means of a cap rotatably mounted to the tubular body with actuating means associated with it. The cap and tubular body have openings of similar dimensions. Rotation of the cap to bring the openings into register causes the actuating means to move the mouthpiece towards an extracted position. Counter-rotation of the cap takes the openings out of register and causes the mouthpiece to be retracted to a closed off position within the tubular body. While the described arrangement seeks to provide a hygienic arrangement for storage of a metered-dose inhaler, it has a number of problems. The first problem arises from the fact that the mouthpiece moves perpendicularly to a long axis of the tubular body when moving to its extracted position. This provides a right angle arrangement between bottom edge of the mouthpiece and the adjacent edge of the tubular body. A user of an MDI typically locates the mouthpiece in his or her mouth by holding the tubular body vertically in front of the person's nose for best alignment with their intake air inhaled by the user. Due to the right angular arrangement described in the present device, this will inevitably lead to tilting of the mouthpiece when it is placed in appropriate proximity to the user's lips so that the direction of the dispensed flume of medication is towards the roof of the user's mouth thereby coating the mucous membranes with the medication rather than entraining it in the inhaled breath.

A further problem arising from the embodiment of FIGS. 5 to 8 of Somova is that the extraction of the mouthpiece relies on the camming means first acting on a rear portion of the mouthpiece followed by completion by the action of the adjacent side edge of the opening 11. This appears to imply that rotation of the cap will push the mouthpiece outwardly in the same way that counter rotation is expected to cause retraction of the mouthpiece. This would appear unlikely as the apertures are of the same dimensions. Hence once the adjacent edge of the aperture is in alignment with the aperture in the tubular body, there is no further sliding activity to cause extraction of the mouthpiece. It is unlikely this camming arrangement would work.

A further problem may arise in that retraction of the mouthpiece is reliant on movement of the rotatable cap causing the edge to slide across the curved mouthpiece to thereby overcome the deploying force. All embodiments show the mouthpiece fitting through the aperture in the outer wall in close approximation with the edges of the outer wall. The mouthpiece and outer wall aperture must be of similar dimensions. Retraction of the mouthpiece will therefore be highly likely to stick and will also require a hard edge to the cap in order to have any chance of operation. The described embodiments will be highly prone to erratic operation, particularly in retraction, and will require a hard cap which is less able to adequately seal the present arrangement.

A further deficiency arises from the fact that there is considerable clear space between a bottom edge of the mouthpiece and the top of the MDI adjacent the protruding stem of the valve. This would permit accidental discharge in the pocket of user particularly when engaged in strenuous activities. Further, because of the present arrangement, there is a considerable space between a rear edge of the mouth piece and the outlet of the MDI discharge valve. This creates a risk of unintended distribution of the spray on the inner walls of the device rather than delivery to the user.

The drive means to extract and retract the mouthpiece lacks a positive, single source of propulsion operating through the full distance of travel. This may cause more operational problems Given the range of rotation of the mouthpiece, if it is not fully deployed, operation of the aerosol container will cause distribution onto a wall of the mouthpiece.

As noted, in inclement circumstances, the risk of contamination may be significant. A sealed, safe and robust storage arrangement would be of advantage, particularly if designed for easy and effective use when required.

Use of therapeutics in emergency situations may present different risks which can be highly serious. Delivery of an analgesic, for example, requires an arrangement that is rotust, reliable and easy to operate. This is particularly the case in the circumstance of self-administration where a user may be, at least partially, incapacitated by injury or people lending assistance may be untrained in first aid.

The provision of a robust, serviceable and effective medication dispenser would be a positive contribution to the quality of life of users. If such a device were to be also compact and even stylish, it would limit or avoid embarrassment associated with its use.

Reference to any prior art in this specification is not, and should not be taken as, an acknowledgement or any form of suggestion that this prior art forms part of the common general knowledge in any country.

### SUMMARY OF THE INVENTION

In a first aspect but not necessarily the only or indeed the broadest aspect, the invention may reside in a medication dispenser for receiving a pressurised medication container and discharging therapeutic doses therefrom, the medication dispenser comprising:

an elongate tubular body having a lateral aperture formed in a first end region;

a cavity formed in the body and adapted to receive the medication container, the cavity in communication with the lateral aperture;

a discharge arrangement for discharging the therapeutic doses;

a rotatable sleeve mounted to the first end region, the sleeve having a sleeve aperture movable by rotation into and out of alignment with the lateral aperture;

a mouthpiece movable between an extended discharge position, extending through the lateral aperture and sleeve aperture when aligned, and a retracted storage position within the cavity;

wherein:

a longitudinal axis of the elongate body deviates away from the position of the extended mouthpiece, the deviation occurring outside the first end region. This is similar to the longitudinal axis deviating away from the lateral aperture The tubular body may be substantially cylindrical.

The pressurised medication container may be any suitable device such as a metered-dose inhaler. Medication in this context should be understood to include any agent which may have a beneficial effect on the user and may be medicinal agents, therapeutic agents or nutrient agents. The medicinal agents may be for the treatment or prevention of disease.

The discharge arrangement may include a hollow seat for receiving the discharge stem of discharge valve in the pressurised medication container. The hollow seat may be positioned in or towards the first end region.

The discharge arrangement preferably further comprises a flexible end cover applied to a second end of the elongated body thereby permitting digital activation of the medication dispenser by pressure. Alternative arrangements such as rotationally activated cammed arrangements may be used to push the container against the hollow seat and discharge the contents. The closure of the second end should be substantially sealed against environmental contamination of the device.

The mouthpiece preferably moves substantially radially between the extended discharge and retracted storage positions.

Preferably the components are dimensioned so that the mouthpiece blocks downward movement of the medication container in the storage retracted position to thereby prevent accidental discharge.

The rotatable sleeve is preferably formed as a closed sleeve or end cap. The sleeve may be formed form a soft and resilient rubber or similar compound which is fitted elastically into place. This provides a readily usable and robust construction which effectively seals the medication dispenser. One alternative suitable material for the sleeve is a silicon or silicon based polymer. An additional advantage in some embodiments may be obtained by the use of self lubricating material such as one incorporating Teflon. The sleeve aperture is preferably considerably wider than the lateral aperture and may be in the range of 25% to 200% wider.

Preferably rotation of the sleeve drives the mouthpiece back and forth between the discharge position and the storage position. One preferred means for so doing comprises a drive arrangement radially spaced from the centre of the end cap and engaged with the mouthpiece to positively drive it between the discharge position and the retracted storage position. The drive arrangement may comprise a pin engaged with a corresponding seat on cooperating components.

Alternatively, the mouthpiece may be driven by a resilient member to the discharge position, with subsequent overriding of the resilient member by rotation of the end cap against a curved surface of the mouthpiece. The resilient member may be a coil spring or an elastic band or other suitable arrangement.

The longitudinal axis of the tubular body preferably deviates between 5° and 20° just outside the first end region. The longitudinal axis preferably deviates in a plane which is diametrically opposite to the position of the mouthpiece when extended. This is similar to diametrically opposite the lateral aperture.

The device may include a locking arrangement for fixing the end cover to the tubular body. The locking arrangement may comprise a lock ring. Additionally or alternatively the device may include a second locking arrangement for locking the medication container to the tubular body. The second locking arrangement may comprise a lock ring, preferably a split ring.

A raised lip may be provided around the lateral aperture to assist in sealing when in the storage position.

The medication dispenser may be adapted to provide an audible signal when the mouthpiece is at full range of movement.

The medication dispenser may further include a container locking arrangement to resist removal of the medication container.

The medication dispenser preferably further includes an electronics system for effecting on more of the following:

(a) count the number of times the medication container and dispenser has been operated;

(b) calculate the number of applications that are left in the container or provide an indication of the number of applications that may be safely used;

(c) calculate the time since the last application;

(d) calculate the number of times used in a set period of time such as 24 hours;

(e) monitor the age of the therapeutic contents of the container;

(f) calculate the dosage dispensed in total or over set times;

(g) provide an alert for moisture or other contaminations inside the dispenser;

(h) provide audible cues on the correct use and duration of operation; and

(i) provide a visible and audible overuse alert.

The electronics system may comprise a processor, power supply, trigger in communication with the processor and a display to display information to a user. The display is preferably a screen display.

The medication dispenser may include a spacer adapter.

The invention may extend to a carrier for a medication dispenser as described above, the carrier comprising a dispenser seat to receive the medication dispenser and a locking bracket for locking the medication dispenser in position. The carrier preferably includes attachment means for attaching to a belt, a strap, an item of clothing or other personal item. The attachment means may be adapted to secure the carrier to a part of a users body such as a limb, around the torso or waist.

The attachment means may be a spring clip belt clip, tongue, eyelet, loops for threading Velcro® or other suitable arrangement.

The dispenser seat is preferably formed with an angular deviation that approximates or mirrors that of the longitudinal axis of the medication dispenser.

The locking bracket may be a saddle which is clipped into locking position and held in position by one or more resilient tabs. The tabs may be activated by pressure to release the saddle.

Preferably the locking bracket is formed with a mounting arm connected to the seat and rotatable into and out of locking position. A tongue may be provided for releasable engagement with a slot, each located on a respective one of the locking bracket and the seat.

The carrier may further include an accessary or accessories comprising one or more of:

(a) a simple mount;

(b) a holder belt clip mount;

(c) a carabiner;

(d) a fixed strap or loop;

(e) a hook and loop fastener;

(f) a lanyard;

(g) a security cap; and

(h) a mount for a bicycle or other equipment.

### BRIEF DESCRIPTION OF THE DRAWINGS

FIG. 1 shows a perspective view of a first embodiment of a medication dispenser of the present invention in storage configuration and discharge configuration;

FIG. 2 shows a sectional view of the medication dispenser taken along the line A-A of FIG. 2A;

FIG. 3 shows a sectional view taken along the line B-B of FIG. 3A;

FIG. 4 shows an exploded view of a preferred embodiment of a medication dispenser of the present invention;

FIG. 5 shows an embodiment including a locking ring shown in a sectional view along line C-C of FIG. 5A;

FIG. 6 shows an embodiment including a locking split ring to resist removal of a medication container shown in a sectional view taken along the line D-D of FIG. 6A;

FIG. 7 shows sectional views of another embodiment of the present invention with FIG. 7B showing a sectional view of FIG. 7A along the line E-E and FIG. 7D showing a sectional view of FIG. 7C along the line F-F;

FIG. 8 is a sectional view of a further embodiment in which FIG. 5B is a sectional view of FIG. 8A along the line H-H;

FIG. 9 is a sectional view of yet a further embodiment in which FIG. 9B is a sectional view of FIG. 9A along the line I-I;

FIG. 10 shows an embodiment including a flexible cover or end cap in which FIG. 10B is a sectional view of FIG. 10A along the line J-J;

FIG. 11 shows perspective views of a cearder for a medication holder in locked and open configurations;

FIG. 12 is a perspective view of the carrier and medication dispenser in combination;

FIG. 13 shows a sectional view of the arrangement of FIG. 12 and taken along the line KK of FIG. 13A;

FIG. 14 is a series of views showing an alternative embodiment of a carrier;

FIG. 15 is an exploded view of a further embodiment of a medication dispenser including an audible signal for open and closed positions;

FIG. 16 is a cross-sectional view of an external locking arrangement for the medication container;

FIG. 17 is a cross-sectional view of an internal locking arrangement for the medication container;

FIG. 18 shows a variety of mounting accessories for use with the medication dispenser and carrier;

FIG. 19 shows two further alternative mounting accessories for use with the medication dispenser and carrier of the present invention;

FIG. 20 shows a isometric view of a dispenser of the present invention with a first embodiment of a spacer;

FIG. 21 is a isometric view of a medication dispenser of the present invention with a second embodiment of a spacer;

FIG. 22 is a schematic diagram of a dispenser with an electronics system;

FIG. 23 is a cross-sectional view of a dispenser with electronics system; and

FIG. 24 shows isometric views of alternate configurations with alternate screen positions for an electronics system.

### DETAILED DESCRIPTION OF THE PREFERRED EMBODIMENTS

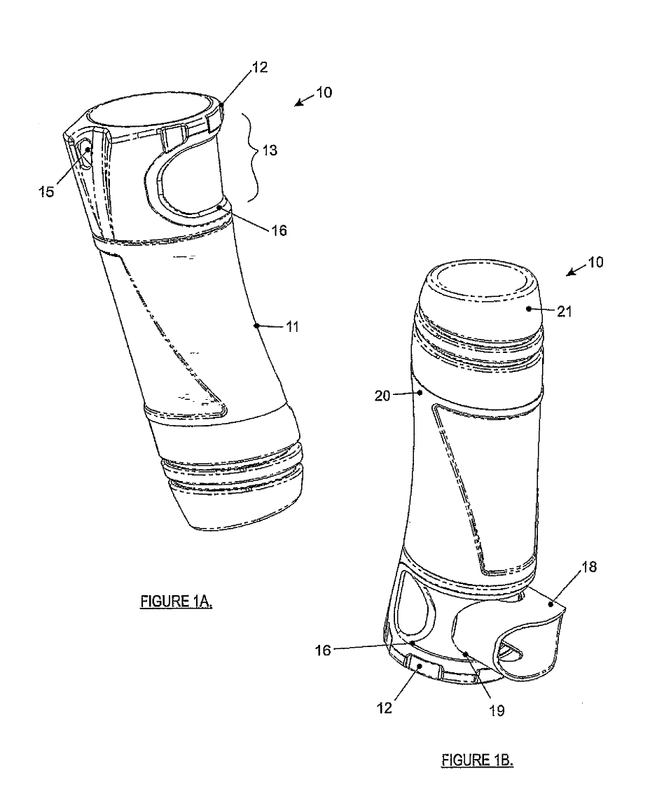

Referring to FIG. 1 there is seen a first embodiment of a medication dispenser 10 shown in FIG. 1A in the storage configuration and in FIG. 1B in the discharge configuration. The medication dispenser comprises a tubular body 11 with a closed sleeve or end cap 12 rotatably mounted to a first end section 13. The sleeve 12 has a mounting aperture 15 to receive a lanyard or similar to be used for example as a wrist strap or neck strap or other securing arrangement. The cap 12 has a sleeve aperture 16 which in the storage configuration overlaps the wall of the tubular body in the first end section 13.

In FIG. 1B a mouthpiece 18 is shown in a discharge or extended position and extends through the sleeve aperture 16 and a lateral aperture 19 of the tubular body 11. The sleeve aperture 16 is considerably longer in its lengthwise dimension than the lateral aperture 19 thereby allowing considerable rotation of the cap 12 with the two apertures overlapping. This allows for movement of the mouthpiece into and out of a full discharge position.

A second end 20 of the tubular body 11 is closed by a deformable rubber cap 21 which may be depressed by the digit of a user to activate the discharge of a medication container located inside the tubular body.

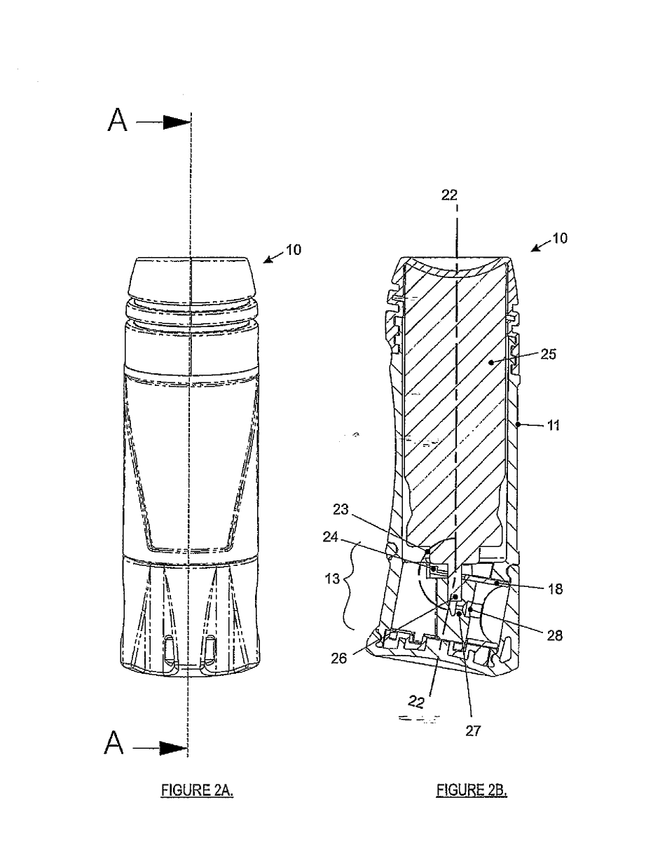

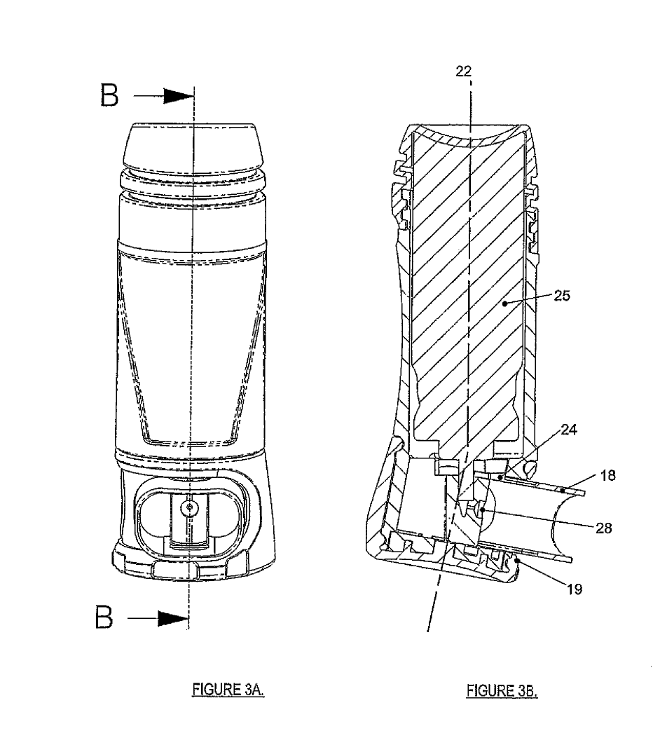

FIG. 2B shows a sectional view of a medication dispenser 10 of FIG. 2A taken along the line A-A. This view highlights retraction of the mouthpiece 18 in the storage configuration. It also shows longitudinal axis 22 of the tubular body 11 with an angled deviation 23 just outside and adjacent the first end section 13. This results in a slightly doglegged effect to the axis 22. The deviation is away from the deployed position of the mouthpiece and also the lateral aperture 19 which is more apparent in FIG. 3 and which allows the user to align the tubular body 11 substantially with the midline of the users body and with the mouthpiece directed to the centre of the mouth enhancing delivery of the therapeutic agent. The deviation allows the tubular body to clear a users nose without directing the therapeutic flume at the roof of the mouth. This arrangement allows the mouthpiece to move laterally in relation to the first end section in a substantially perpendicular fashion, thereby simplifying the drive arrangement and enhancing its reliability. The angle of deviation is preferably in the range of 5-20 degrees although other angles may be suitable.

Another advantage of the present arrangement is seen in FIG. 2B wherein retraction of the mouthpiece 18 leads to a rearward edge and upper wall 24 angling upwards relative to the medication container to obstruct downward movement of the medication container when the mouthpiece is retracted. This prevents accidental discharge through the valve 26 by inadvertent downward pressure on the container 25. The valve 26 sits into a seat 27 formed in the tubular body 11. The seat is hollow to communicate with and discharge through outlet 28 (seen in FIG. 3).

The rearward edge and upper wall 24 is seen moved clear of the container 25 when the mouthpiece 18 is deployed as seen in FIG. 3.

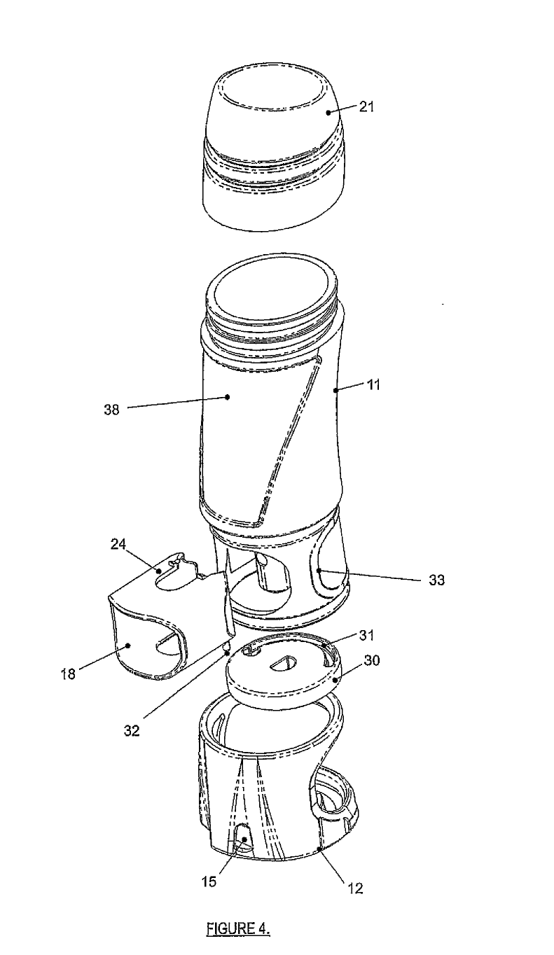

FIG. 4 shows exploded components comprising tubular body 11, cap 21, mouthpiece 18 and closed sleeve or end cap 1 2.This view also shows a plate 30 with a circumferential slot 31 which engages a pin or drive pin 32 on the mouthpiece 18 when assembled. Rotation of the cap 12 causes rotation of the plate 30 which in turn drives the pin 32 and mouthpiece 18 inwards or outwards according to the direction of rotation.

This embodiment also shows a raised ring 33 which enhances engagement between an inner wall of the closed sleeve 12 and the tubular body 11 when in the storage position. This raised ring further and better protects the enclosed and sealed medication container when not in use. This view also shows the mounting aperture 15.

FIG. 4 also shows a semi-transparent window 38 which allows visualisation of the medication container. The window may be transparent to allow full appreciation of a label of the contents.

FIG. 5 shows an embodiment including a locking ring 35 co-operating with the cap 21 to fix it to the tubular body 11 and thereby resist tampering. The locking ring has two spaced ribs 36, 37 which engage corresponding grooves in the tubular body 11 and cap 21 respectively to resist separation. It may also act as an impediment to reuse of the device in the event that it is considered preferable to dispose of the medication device entirely (including the medication dispenser) at the end of one complete cycle of use.

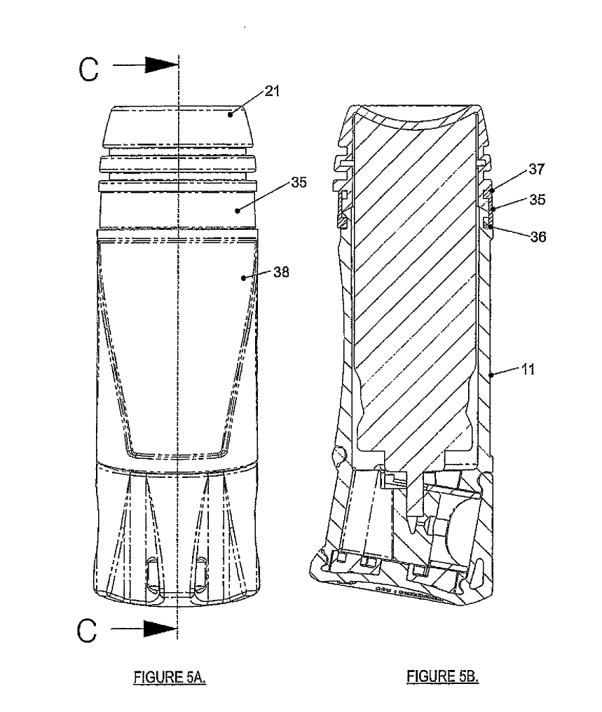

In FIG. 6 a locking split ring 34 is shown which may be used to lock the medication container 25 in position and thereby also resist tampering and/or replacement in the event that multiple re-use of the device is not required. The split ring has a shoulder 39 that sits into a corresponding recess in the tubular body 11 and then abuts the medication container to resist its upward movement.

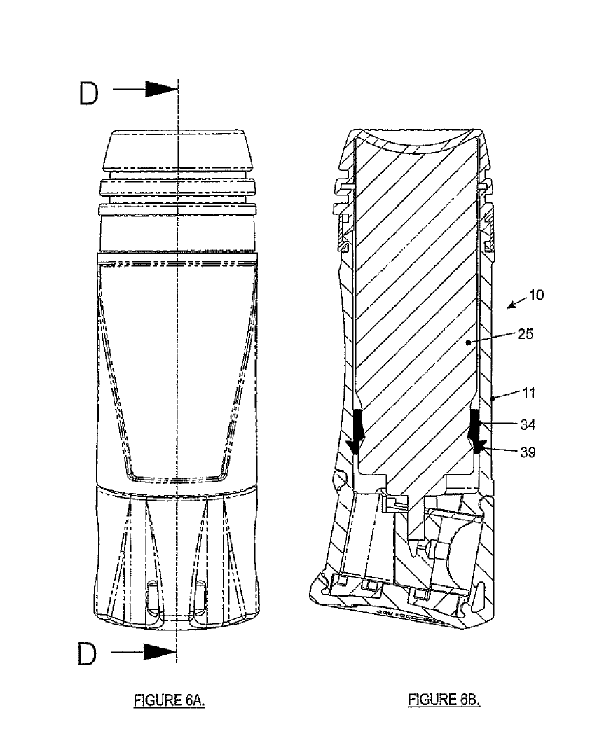

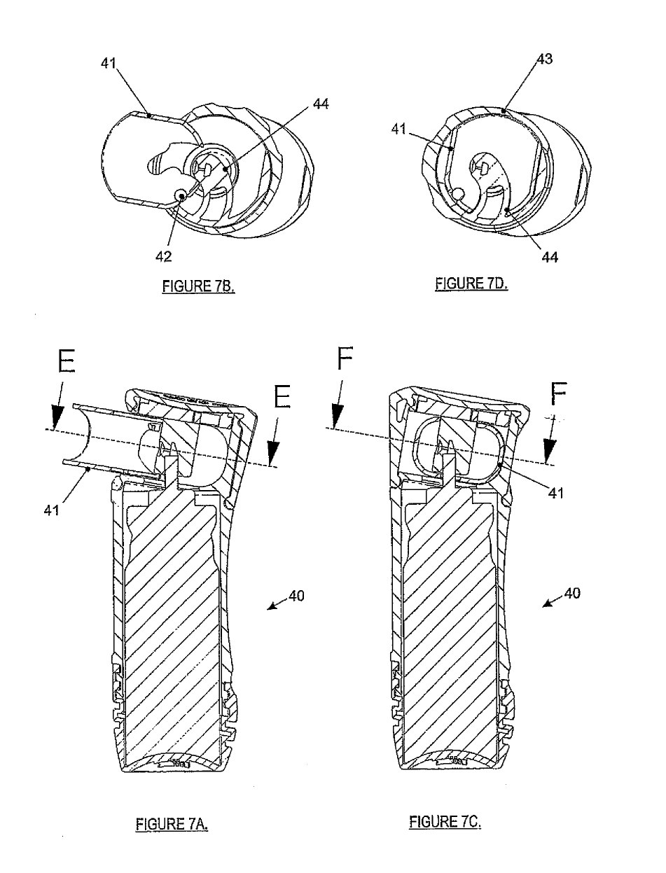

FIG. 7 shows an alternative embodiment of a medication dispenser 40 with FIGS. 7A and 7B showing side and top sectional views respectively in the discharge configuration and FIGS. 7C and 7D showing side and top sectional views in the storage configuration. In this embodiment the mouthpiece 41 rotates around a pin 42 to rotate through approximately 90° to the storage position seen in FIGS. 7C and 7D or likewise in reverse. Rotation of the cap 43 causes the mouthpiece 41 to automatically slide out along curved guide 44 into an extended position. Reverse rotation causes the mouthpiece to be retracted.

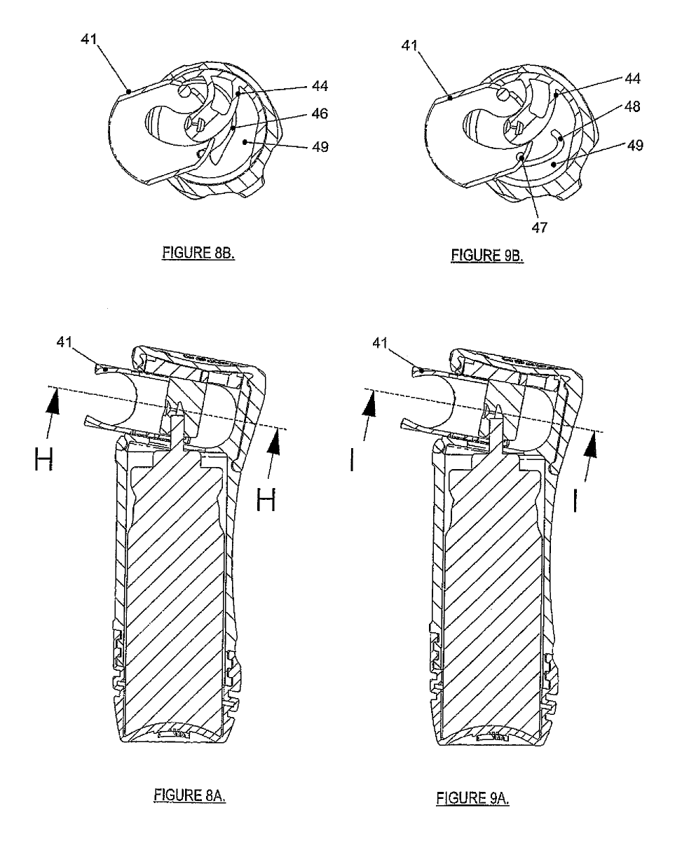

FIG. 8 shows a similar arrangement to that of FIG. 7 but including a spring 46 which urges the mouthpiece 41 outwardly. The resistance of the spring is overcome by counter rotation of the device causing the mouthpiece to retract around the curved guide 44.

FIG. 9 shows an arrangement in FIG. 9B in which a cam drive is effected by the interaction of drive pin 47 and slot 48. The curved guide 44 allows the mouthpiece 41 to rotate and nest snugly within the tubular body in the retracted configuration.

The views in FIGS. 8B and 9B are taken along the arrows H-H and I-I respectively and are upward looking views. The plate 49 is also apparent in these views.

FIG. 10 shows an embodiment of a medication dispenser 50 with FIG. 10B being a sectional view of the embodiment of FIG. 10A taken along the line J-J. This embodiment shows the addition of a flexible cover 51 which overlaps the mouthpiece so that extrusion or retraction causes the edges and outer walls of the mouthpiece 52 to be automatically wiped thereby enhancing cleanliness.

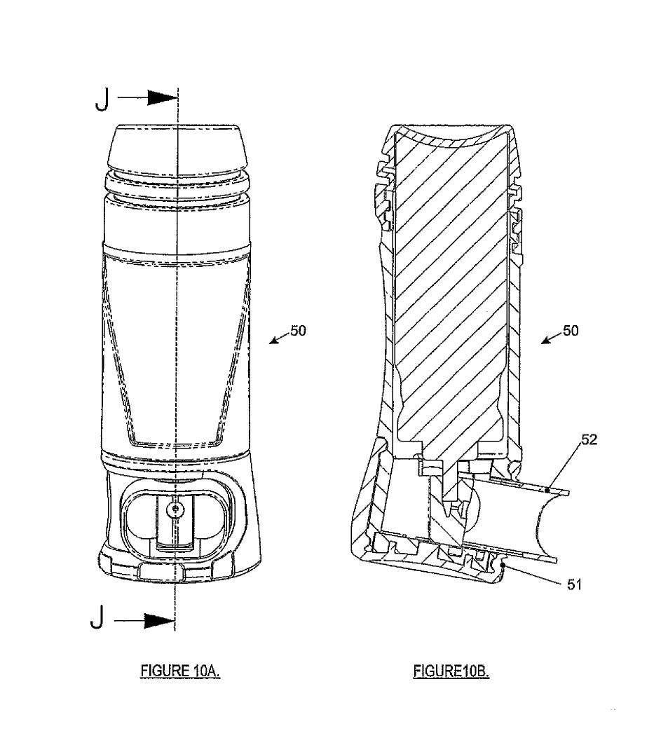

FIG. 11 shows a perspective view of a carrier for the medication dispenser with FIG. 11A showing the carrier with locking arm closed and FIG. 11B with the locking arm open to receive the medication dispenser. The carrier comprises a dispenser seat 61 for receiving a medication dispenser The dispenser seat 61 comprises a lower floor 62 and semi-encircling wall 63. Advantageously the wall 63 is curved or angled to conform with the angular deviation of the body of the medication dispenser. A locking bracket is formed as a bail arm 64 which is hingedly mounted at a top hinge 65 and has a locking tongue 66 adapted to snap into receiving slot 67 as seen in FIG. 11A. This is however a non limiting example of the locking bracket.

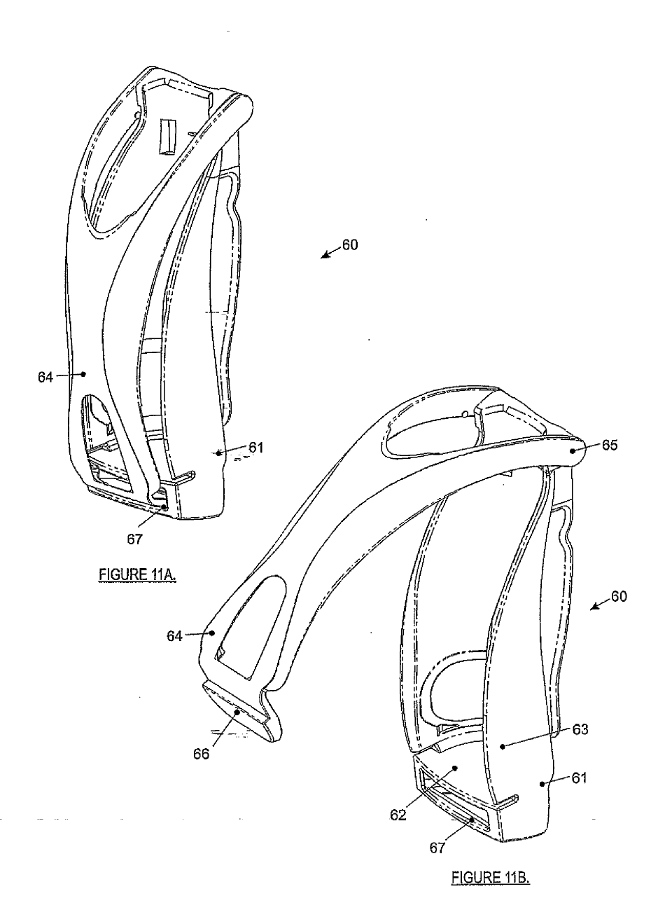

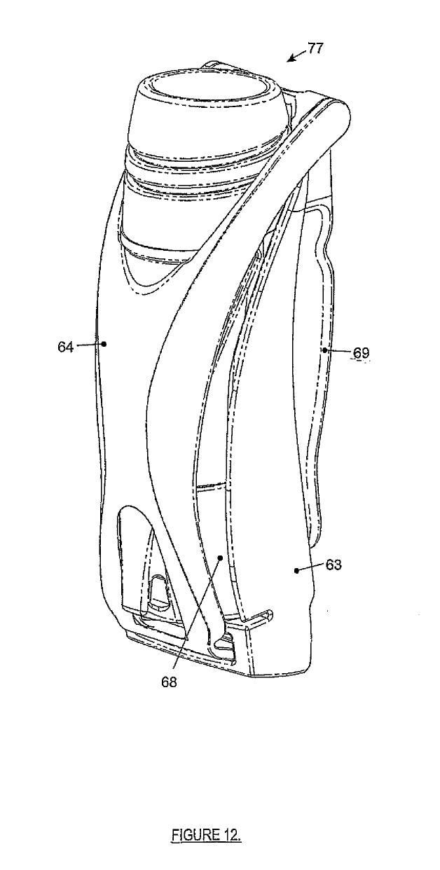

FIG. 12 shows a medication dispenser 77 in position with the bail arm 64 locked down. The carrier is preferably dimensioned so that the bail arm 64 applies a slightly compressive force against the medication dispenser 77. One particular advantage of matching the shape of the wall 63 to that of the dispenser 77 is that it naturally orientates the lateral aperture of the end cap 68 to the back of the carrier and into the wall thereby leading to a snug storage which adds an extra layer of protection against environmental contamination. Further, the matching contours lead to an increased resistance to the dispenser rising vertically as would be possible if it was a simple tubular body with no deviation. As is apparent in the views the carrier has a slot formed by rearward tongue 69 which extends downwardly and allows slipping of the carrier over an item such as a belt. Alternatively a loop may be formed or any suitable mounting means for application to an item of clothing, a strap for attachment to a person or the strap of a rucksack, haversack, backpack, sportsbag or any application which would be envisaged by a person skilled in the art.

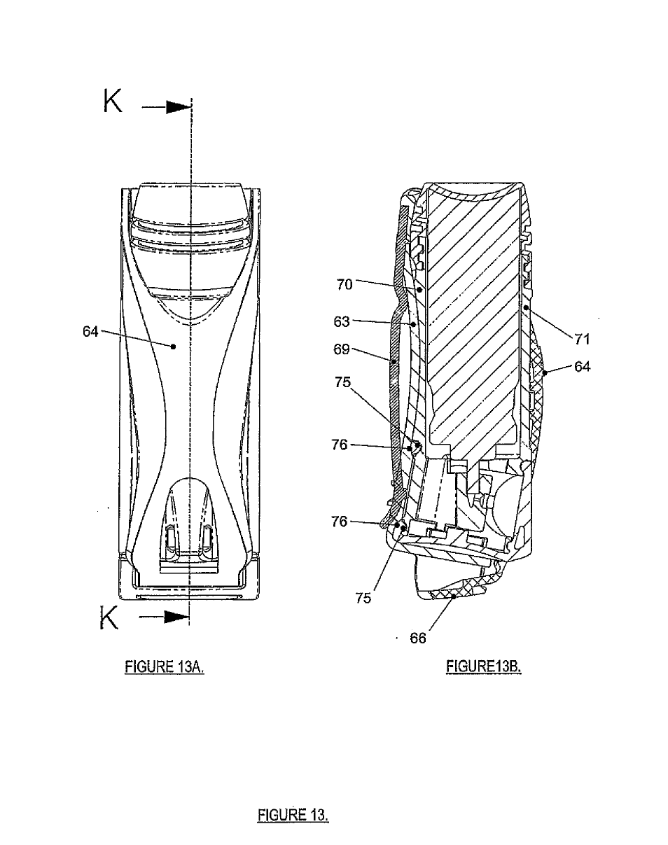

FIG. 13 shows a forward view of the arrangement of FIG. 12 in FIG. 13A and a sectional view of that arrangement in FIG. 13B taken along the lines K-K. It is apparent the wall 63 conforms closely to the wall 70 of the tubular body 71 at the rearward portion and anterior to the tongue 69. The bail arm 64 is fitted snugly against the tubular body 71 to urge it backwardly and the angle of deviation of the longitudinal axis and the carrier match to restrict the tendency for the device to be dislodged. The locking tongue 66 is inserted into the slot to hold the device in position.

A further feature of this view is two recesses 75 formed to receive ring structures 76 in the rotatable cap to better seal the join between the two components. An alternative arrangement may include a raised lip or even a step, dimensioned to allow an edge of the sleeve aperture to slide over or onto it and nest securely against it.

Of course other locking means may be used. For example a saddle bracket may clip into place across the tubular body and lock against one or more spring tabs which are resiliently snapped into locking position. Such an embodiment is seen in the carrier of FIG. 14. A carrier 81 is formed with a curved side wall 82 and floor 83. The carrier 81 has a tongue loop 84 for receiving a belt or similar. A saddle bracket 85 has paired slots 86 on either side. The slots are adapted to clip over respective mating pins 87 located on the side wall 82. The bracket may be released by depression of the tabs 88 to allow clearance of the device and removal of the medication dispenser. In use the saddle bracket travels in and out along the length of the slots 86.

The angled longitudinal axis also allows instant and automatic orientation of the device for use so that it may be used in low illumination situations with confidence.

As noted, the present device may include a transparent panel to allow visualisation of the medication container and review of information printed thereon.

The medication dispenser and/or the carrier may be formed, at least in part, from polypropylene, polyethylene and/or nylon. Other materials may be used and, indeed metals and metal alloys may be suitable. A particularly robust, hardwearing and hygienic device may be formed from stainless steel.

The dispenser and carrier are preferably formed from an injection moulding process.

FIG. 15 shows an exploded view of a dispenser 110 with a deformable rubber cap 121, tubular body 111, mouthpiece 118, and sleeve 112. The plate 130 is provided for driving the mouthpiece 118 through pin 132. The plate 130 is shown in close up having a drive slot 131. A secondary slot 133 is provided with small deformable teeth 134 at either end. The slot is adapted to receive a lug positioned on the body which travels in the secondary slot. At either end, the lug distorts the teeth which click back into place once the lug has finished its allowable travel thereby providing an audible click sound at the end of travel in either direction. This lug (not shown) may also act as a secondary stop arrangement for the travel of the mouthpiece.

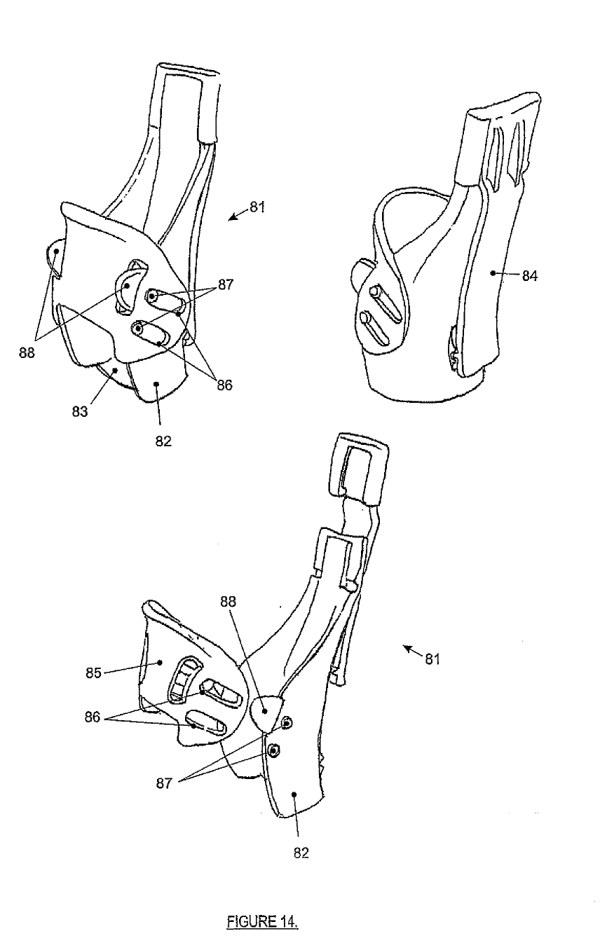

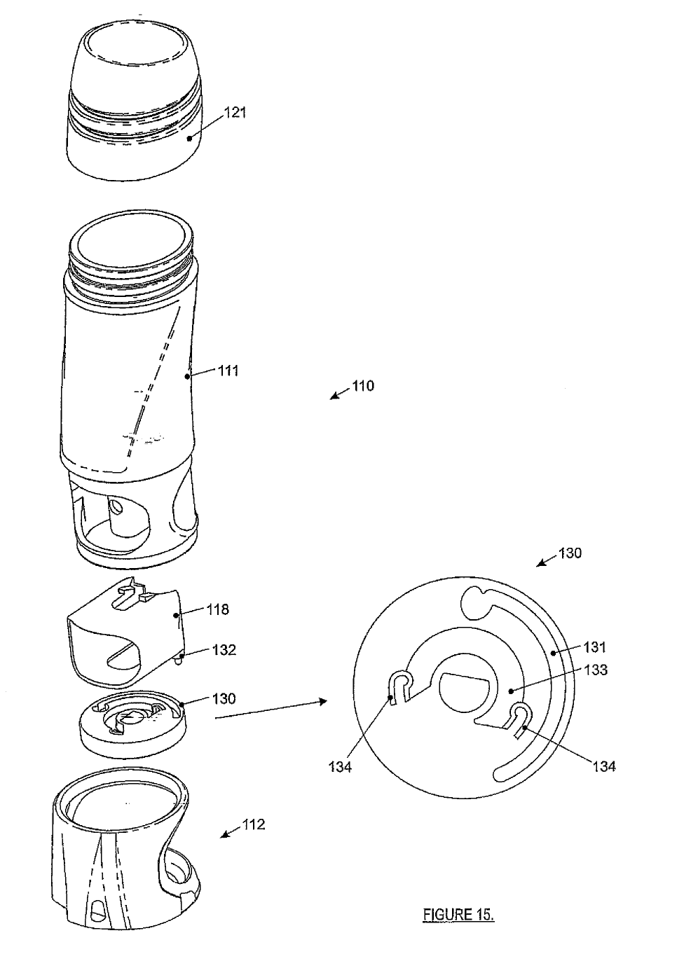

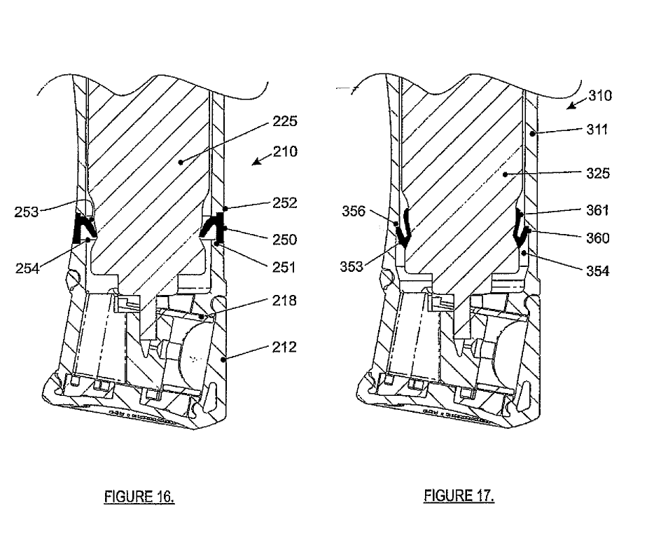

FIGS. 16 and 17 show two alternative arrangements for locking the medication container in position in the medication dispenser. These may be used as a security arrangement to prevent dislocation of the container. They may also be used for single use arrangements to ensure dispensers are not recycled in conditions where hygiene and drug efficacy require regular turn over of the device. In FIG. 16 a dispenser 210 has a medication container 225 in position with a retracted mouthpiece 218. The end cap or sleeve 212 is rotated to a closed position. An external “C” clip 250 is positioned in a receiving groove 251. The C clip comprises an external wall 252 with an inwardly extending rib 253 which is angled inwardly and upwardly relative to the container 225 and engages a groove 254 in the container 225. This location and arrangement resists or will prevent removal of the container 225 from the dispenser 210.

An alternative arrangement is shown in FIG. 17 where a locking split ring 360 is positioned internally in a medication dispenser 310. The split ring 360 has a base 361 closely positioned to the wall of the container 325 and in the groove 354. The base supports a rib 353 which engages a ledge 356. The inside wall of the tubular body 311. This arrangement again resists removal of the container 325 from the dispenser 310.

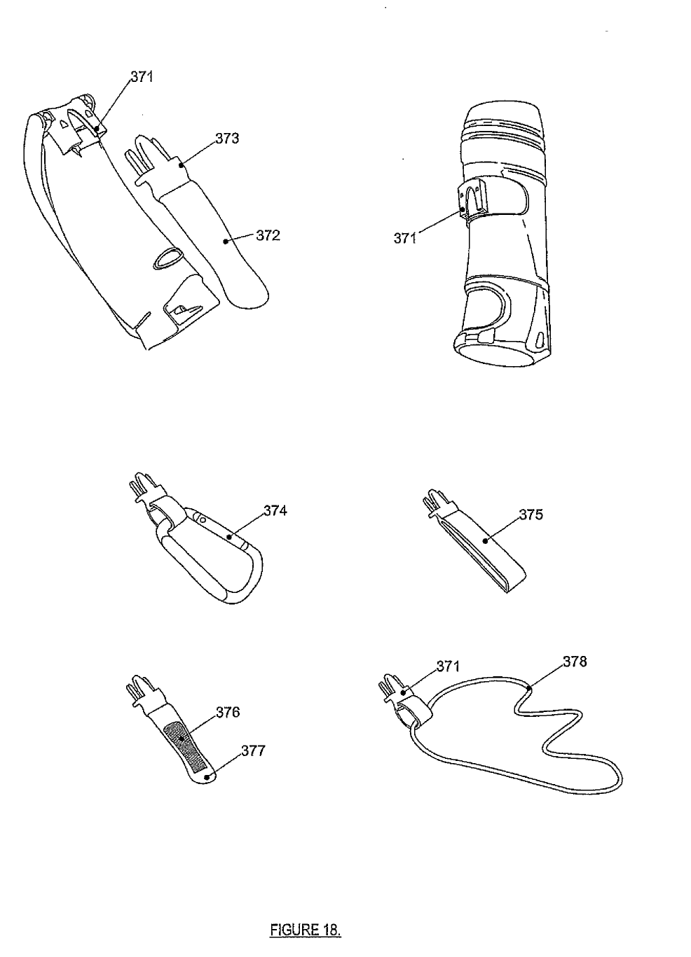

FIGS. 18 and 19 show a variety of add-on accessories for receiving the carder of the present invention and hence for use with the medication dispenser. Those accessories may include a simple mount 371 for engaging a receiving tongue for a device arranged on another item such as a belt. An example of such a device is shown as a holder belt clip-mount 372 in which the clip 373 is adapted for engaging the mount 371. The remainder of the device may be a loop for receiving a belt. An alternative arrangement including a carabiner 374 is shown. A fixed belt/strap may be used 375. A Velcro® band 376 may be placed upon the belt 377 and adapted the location around the limb of a user or other positioning. A lanyard 378 can be used with the single mount 371. A security cap 379 is shown in FIG. 19 with the safety lanyard 380 which is fixed to tongue 381 of the carrier 382. Security cap 379 enhances the closure of the carrier by locating the lower region and preventing dislodgement of the locking arm of the carrier. A mounting arrangement 384 for a bicycle is shown with a base mounting plate 385 attached to a mounting bracket 386 to receive the dispenser and carrier 387. The base mounting plate 385 may be slipped into contact with a suitably sized tongue and the bracket 386 supports the device while also securing the carrier.

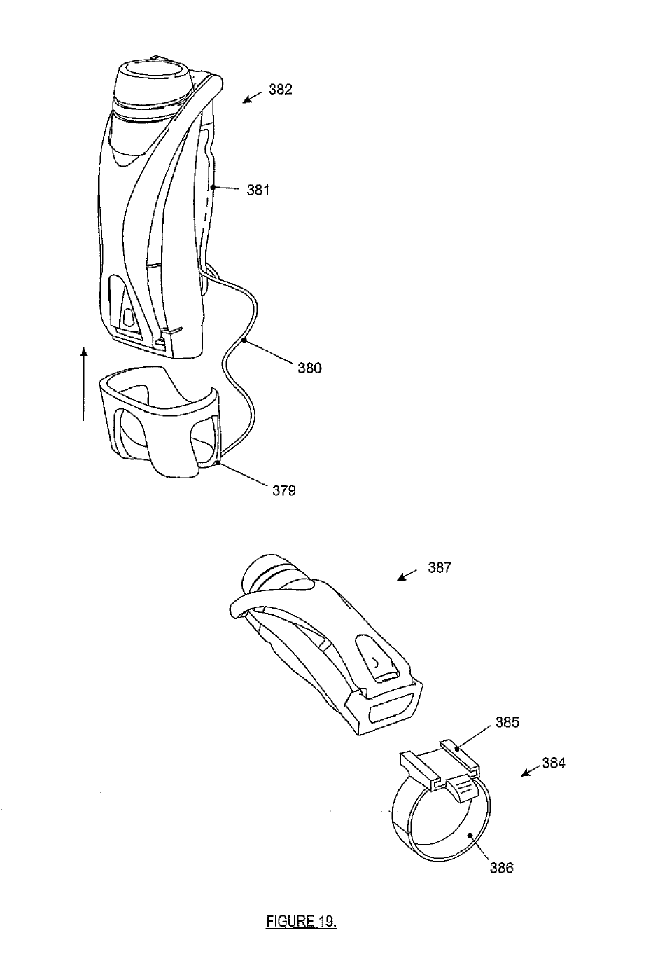

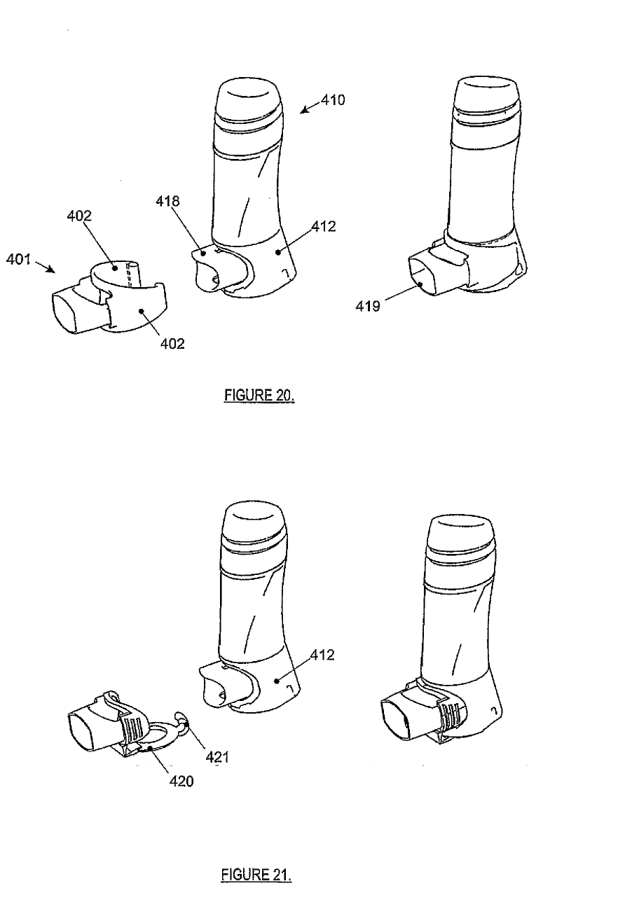

The present invention may also include a spacer adapter to permit use with a known spacer. Spacers are provided to enhance the transfer of propelled medication into a user's body. The spacer provides an air volume into which the medication is discharged allowing for prolonged inhalation by a user. As the dimensions of the present medication dispenser, particularly the mouthpiece, may be scaled down for efficient storage and use, a spacer adapter may be required. FIG. 20 shows a spacer adapter 401 for use with a medication dispenser 410 by snap fitting of two arms 402 around the end cap 412 to sit over the mouthpiece 418 and provide an enlarged outlet 419 dimensioned to engage the spacer.

An alternative arrangement is shown in FIG. 21 where attachment is achieved by resilient band 420 with a hook 421. This is positioned over the back of the end cap 412 to provide the same effect.

FIG. 22 is a schematic view of an electronics system for use in a medication dispenser of the present invention. An electronics system 500 is provided comprising a mechanical trigger input 501. This trigger is preferably waterproof and is activated when the medication container is depressed. The trigger 501 is in communication with a processor 502 which includes a timer and also a counter function. The processor is driven by a power source 503 which is preferably a battery.

The processor is programmed to carry out preferred operations which may include one or more of the following:

(a) count the number of times the medication container and dispenser has been operated;

(b) calculate the number of applications that are left in the container or provide an indication of the number of applications that may be safely used;

(c) calculate the time since the last application;

(d) calculate the number of times used in a set period of time such as 24 hours;

(e) monitor the age of the therapeutic contents of the container;

(f) calculate the dosage dispensed in total or over set times;

(g) provide an alert for moisture or other contaminations inside the dispenser;

(h) provide audible cues on the correct use and duration of operation; and

(i) provide a visible and audible overuse alert.

The processor displays results on a output interface 504 and may include graphic displays of preferred information. This preferred information may contain total use count diagrams, time since last use and a warning when levels are low, amongst other information. It may also issue audible and visual alarms if the device is over utilised or if a set period of time has lapsed since the last application and it is time for a top-up. Other information and processes may be effected by the electronic system.

FIG. 23 shows the position of the processor and power source 510 and a switch 501 for manual operation by depression of the end cap 521 and displacement of the medication container 525. A shoulder 526 of the container 525 pushes on the switch 501 to activate a recording by the processor 502. The switch may be electronic in function.

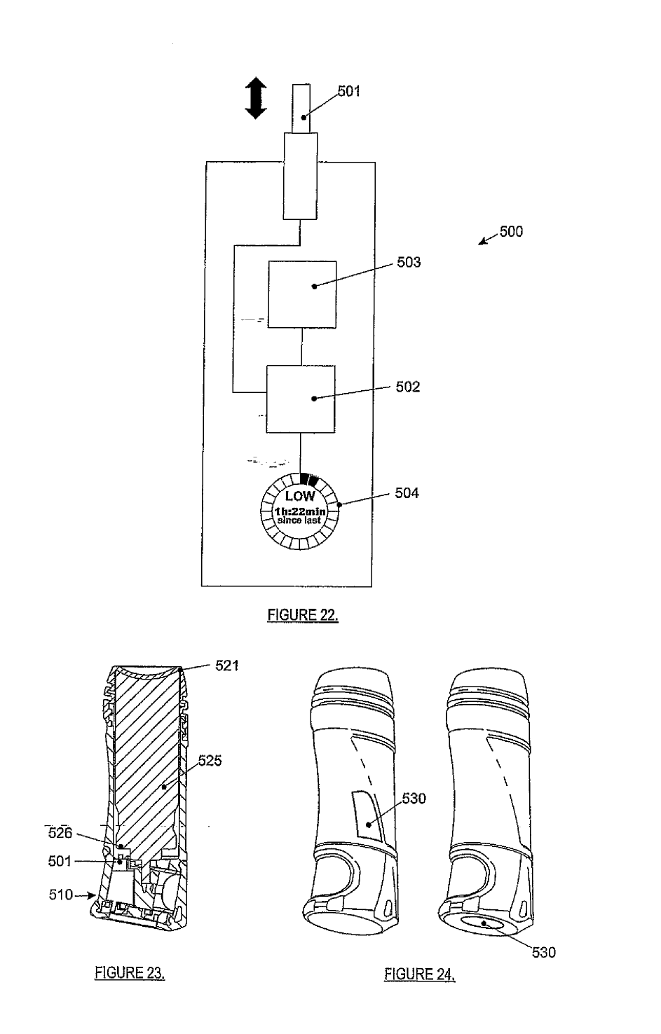

FIG. 24 shows alternative positions for a screen 530 in the medication dispenser for easy access by a user.

The above detailed description provides preferred exemplary embodiments only, and is not intended to limit the scope, applicability, or configuration of the present invention. Rather, the detailed description of the preferred exemplary embodiments provides those skilled in the art with an enabling description for implementing the preferred exemplary embodiments of the invention. It should be understood that various changes can be made in the function and arrangement of elements and steps without departing from the spirit and scope of the invention.

1. A medication dispenser for receiving a pressurised medication container and discharging therapeutic doses therefrom, the medication dispenser comprising:
an elongate tubular body having a lateral aperture formed in a first end region;
a cavity formed in the body and adapted to receive the medication container, the cavity in communication with the lateral aperture;
a discharge arrangement for discharging the therapeutic doses;
a sleeve rotatably mounted to the first end region, the sleeve having a sleeve aperture movable during rotation, into and out of alignment with the lateral aperture;
a mouthpiece movable between a discharge position extending through the lateral aperture and sleeve aperture when aligned and a storage position in the cavity;
wherein:
a longitudinal axis of the elongate body deviates away from the position of the extended mouthpiece in the discharge position, the deviation occurring outside the first end region.

2. The medication dispenser of claim 1 further including the pressurised medication container.

3. The medication dispenser of claim 2 wherein the discharge arrangement includes a hollow seat for receiving a discharge stem of a discharge valve in the pressurised medication container and a flexible end cap applied to a second end of the elongate body permitting digital activation of the medication dispenser.

4. The medication dispenser of claim 2 wherein the tubular body is substantially cylindrical and the mouthpiece moves radially between the discharge position and storage position.

5. The medication dispenser of claim 2 wherein the mouthpiece blocks downward movement of the medication container in the storage position.

6. The medication dispenser of claim 2 wherein the sleeve is formed as an end cap preferably from a soft and resilient material.

7. The medication dispenser of claim 6 wherein the sleeve aperture is in the range of 25 to 200% wider than the lateral aperture.

8. The medication dispenser of claim 2 wherein rotation of the sleeve drives the mouthpiece between the discharge position and the storage position.

9. The medication dispenser of claim 8 wherein a drive arrangement for driving the mouthpiece comprises a pin engaged with corresponding seat, the pin located on the mouthpiece and the seat located on a drive plate and radially spaced from the centre of the drive plate wherein rotation of the end cap causes rotation of the drive plate to urge the pin and thereby the mouthpiece inwards and outwards depending on the direction of rotation.

10. The medication dispenser of claim 2 wherein the mouthpiece is driven by a resilient member to the discharge position with subsequent overriding of the resilient member by rotation of the end cap against a curved surface of the mouthpiece.

11. The medication dispenser of claim 1 wherein the longitudinal axis of the tubular body deviates between 5 and 20°.

12. The medication dispenser of claim 1 wherein the longitudinal axis deviates in a plane which is diametrically opposite the mouthpiece when in the discharge position.

13. The medication dispenser of claim 1 further including a locking arrangement for fixing the end cover to the tubular body.

14. The medication dispenser of claim 1 further comprising a raised lip around the lateral aperture to assist in sealing when in the storage position.

15. The medication dispenser of claim 1 further adapted to provide an audible signal when the mouthpiece is at full range of movement.

16. The medication dispenser of claim 2 further including a container locking arrangement to resist removal of the medication container.

17. The medication dispenser of claim 1 further comprising an electronic system for effecting one or more of the following:
(a) count the number of times the medication container and dispenser has been operated;
(b) calculate the number of applications that are left in the container or provide an indication of the number of applications that may be safely used;
(c) calculate the time since the last application;
(d) calculate the number of times used in a set period of time such as 24 hours;
(e) monitor the age of the therapeutic contents of the container;
(f) calculate the dosage dispensed in total or over set times;
(g) provide an alert for moisture or other contaminations inside the dispenser;
(h) provide audible cues on the correct use and duration of operation; and
(i) provide a visible and audible overuse alert.

18. The medication dispenser of claim 17 wherein the electronic system comprises a processor, a power supply to power the processor, a trigger in communication with the processor and a screen to display information from the processor to the user.

19. The medication dispenser of claim 1 further including a spacer adapter.

20. The medication dispenser of claim 1 in combination with a carrier for the medication dispenser.

21. The medication dispenser and carrier of claim 20 wherein the carrier comprises a dispenser seat to receive the medication dispenser and a locking bracket for locking the medication dispenser in position.

22. The medication dispenser and carrier of 21 wherein the dispenser seat is formed with an angular deviation which approximates or mirrors that of the longitudinal axis of the medication dispenser.

23. The medication dispenser and carrier of claim 21 wherein the locking bracket is a saddle adapted to be clipped into locking position and held in position with one or more resilient tabs.

24. The medication dispenser and carrier of claim 21 wherein the locking bracket is formed with a locking arm rotated into locking position with a tongue releasably engaged with a slot to lock the arm in position.

25. The medication dispenser and carrier of claim 21 wherein the carrier further includes an accessory or accessories comprising one or more of:
(a) simple mount;
(b) holder belt clip mount;
(c) a carabiner;
(d) fixed strap or loop;
(e) a hook and loop fastener;
(f) a lanyard;
(g) a security cap; and
(h) a mount for a bicycle or other equipment.
